(19) United States
(12) Patent Application Publication
(10) Pub. No.: US 2011/0238002 A1
(43) Pub. Date: Sep. 29, 2011

(54) DEVICE FOR PHOTO-DYNAMIC THERAPY OF A LIVING ORGANISM'S TISSUES

(76) Inventor: Abdula KURKAYEV, Budapest (HU)

(21) Appl. No.: 13/019,993

(22) Filed: Feb. 2, 2011

(30) Foreign Application Priority Data
Mar. 25, 2010 (AT) GM196/2010

(51) Int. Cl. A61M 37/00

(52) U.S. Cl. 604/21; 604/20

(57) ABSTRACT

Devices for photo-dynamic therapy of a living organism's tissues comprising an optical light exposure system (I) containing at least one source of light (2) selected from a group including light sources of high power and a large range of wavelength, single LEDs, LEDs in a matrix-arrangement, halogen light sources, laser diodes and laser light sources, a suspension supply system (II) and a system (III) for simultaneous delivery and irradiation of a suspension of a photosensitizer, wherein a central control system (1) is connected to suspension supply system (II) and to a control unit (3) of at least one source of light (2) of the optical light exposure system (I) and enables a simultaneous or a separate operation of both systems (I) and (II).

### BACKGROUND OF THE INVENTION

1. Field of the Invention

A device for photo-dynamic therapy of a living organism's tissue comprises an optical light exposure system consisting of at least one source of light, a photosensitizer supply system, and a system for a simultaneous or separate administration and irradiation of a suspension of a photosensitizer. The device is applicable in human and veterinary medicine for the purpose of treatment of malignant neoplasm, aesthetic defects, different skin and mucous tunic diseases, wounds and ulcers.

2. Description of the Prior Art

In the common photo-dynamic therapy, a photosensitizer molecule is converted into an excited condition after absorbing a quantum of light. Being in that excited condition, it initiates two different chemical reactions. Under the first reaction type, a direct interaction of the excited molecule and molecules of a biologic substrate results in generation of free radicals. Under the second reaction type, the excited photosensitizer interacts to a molecule of so-called triplet oxygen (common oxygen in tissue cells) resulting in catalysis of production of singlet oxygen. Singlet oxygen possesses a huge oxidation potential, which is greater than that of ozone. Under both reaction types, products generated as a result of the photochemical reaction exert a suppressing impact on diseased cells of a living organism and destroy them in that way. (Prof. Dr. Tamas Vidoczi, Differentiation of a photo-dynamic action, Magyar Kémiai Folyóirta, Volume 113, 2007, pages 44-48).

A method of photo-dynamic therapy for treatment of superficially located ulcers of a mouth mucous tunic is known. The known method describes application of a suitable 5-aminolevulinic acid-containing photosensitizer. (Department of pathology, Norwegian Radium Hospital, University of Oslo, 1997, Jan. 15, 1979 (12), 2282-308.

Another device for photo-dynamic therapy (PDT) is known from RU 2 221 605 dd 20.01.2004. The device is used for therapy of malignant ulcers with application of photosensitizers that contain a tumor-tropic agent. It is a common practice in PDT first to administer a suitable photosensitizer to a recipient, then to wait for a time span until the level of photosensitizer rises to an acceptable concentration in a target treatable area and finally to irradiate the target area with a suitable source of light. Molecules of photosensitizers in a target area absorb rays of light and give rise to luminescence and photochemical reactions in the treated area, destroying affected tissues. That device comprises a diagnostic module, which consists of a pathologic topology determiner, a picture saver, an action topology determiner, a display system for pathologic topology data and action topology data, and a light exposure system. The light exposure system comprises sources of laser light, a time management system, and an optical instrument guiding rays of light into a target area. The diagnostic module serves to select irradiation modes and control the diagnostic module. The pathologic topology determiner comprises a video camera, which is equipped with a selective optical instrument that has a spectral window transparent to the luminescence range of the photosensitizer. The biggest photo-dynamic effect is reached, when the wavelength range of the irradiated light corresponds to the photosensitizer's spectral absorption maximum.

Another device for activating a PDT-agent is known from RU 2 371 216 dd. 27.10.2009. The device comprises a storage cavity for storing a PDT-agent, whereas an optical instrument also coupled to a source of light is fixed on a wall of the storage cavity. The storage cavity comprises a cover which has a switch closing a current circuit, when the cover is open, and thereby activating sources of light. A suitable source of light irradiates a PDT-solution (a photo-active agent) directly prior its administration onto a target area. PDT-solution is activated in that way and it is converted from the photo-active condition into a photo-activated one, and reveals thereafter new useful bioactive properties. Then the photo-activated PDT-solution is brought onto a target area, where it exerts its therapeutic and/or cosmetic action. Photo-active PDT-solutions are available in the market in different forms: true, colloid and micellar solutions, gels, creams, mono- and poly-dispersed systems, emulsions, vesicular and liposome systems etc. The known method provides for application of an already photo-activated solution in its bioactive condition.

The device in that form is used both as a vessel for storing a photo-active PDT-solution and as a device for photo-activating that PDT-solution through exposure to light irradiation. For that purpose, a source of light is located directly on the storage cavity that stores the photo-active solution. The device comprises further a power supply unit that provides power for the source of light. The device irradiates light necessary to photo-activate a PDT-agent in the way, that light irradiation evenly influences a photo-active solution in a suitable compartment of the storage cavity directly prior supplying to a target area. A source of light is selected from a group containing incoherent and coherent, monochromatic and non-monochromatic, constant and pulse sources of light of UV-range (from 280 nm to limit of visible range), of visible range and of IR-range (from 840 nm to 1100 nm and from 7000 nm to 14000 nm, respectively). Selection of light sources and modes of light irradiation depends on the type of applied PDT-solution. Prior to the process of photo-activating, PDT-solution is kept in a suitable compartment of the storage cavity, i.e. out of a target area of a living organism; therefore influence on the photo-activation process by tissues is prevented. So, the effect of the PDT procedure is increased and it provides for a new way to apply as PDT-solutions substances that were known to be inactivatable inside the tissues. Light irradiation activates not only photo-active ingredients of a PDT-solution, that are transformed upon exposure to light into therapeutically or cosmetically effective compounds, but also those that serve as auxiliary ingredients to the solution, e.g. compounds and/or structures are used as vehicles for delivering solution deeply into tissues over superficial bounds of tissues (e.g. a trans-dermal vehicle).

It is a disadvantage of the known PDT-method that it requires activating a PDT-solution away from tissues of a living organism, as it limits its beneficial effect on the tissues.

A PDT effect depends on simultaneously administering a suspension of a photosensitizer and exposing it to laser light irradiation, as the life span of singlet oxygen amounts to just some milliseconds.

An embodiment of a PDT device under the name of SCHALI-LAS is known, that comprises an optical channel (light irradiation) and a flowing suspension channel (delivery of a PDT-solution) (see: Nanotechnologies for medicine, A. Kurkayev, Budapest 2008, p. 75). The device comprises a Y-shaped body, wherein one arm is used to supply a flowing photosensitizer into the main channel of Y-shaped body, and the other arm is used to supply light from a light source into the main channel of Y-shaped body, preferably by means of a suitably selected and attached optical fiber. Preferably, the flow rate of the suspension of photosensitizer flowing into the main channel of the Y-shaped body is selected in accordance with the light intensity supplied by the optical fiber. The device also comprises a catheter, an injection needle or a sprayer, dependant on the way of supplying a photosensitizer into a target area. The device provides for photosensitizer supply into a target area and a simultaneous activation thereof, wherein a photosensitizer in the flowing form acts as an optical light conductor for light rays. Nanoparticles of heterocrystal minerals are selected as a photosensitizer. Preferably nanoparticles of Rutil, Sphene (Titanit), Loparite, Perowskite, Anatase, Ilmenite, Leukoxen, Ferrite, Berite, Argyrite, Graphite and Silicon. Nanoparticles of those heterocrystal minerals contain oxides of silicon, titanium and iron. Nanoparticles of that embodiment are used in the form of a transparent suspension, which serves as a kind of light conductor (EP1779855 dd. 28.10.2005).

Schali AG developed a series of pharmaceuticals in the form of stabilized SiO₂ and TiO₂w nanosuspensions. As a nanosuspension, a stable aqueous suspension of the above-mentioned dioxides is understood. The pharmaceuticals are natural products. In all the processing stages of natural raw minerals, mechanical and electro-thermal technologies are applied, preserving the natural initial nanocrystalline structure and properties of minerals (SCHALI AG, Mauren Fla.).

However, operation of SCHALI-LAS device requires a high qualification level of a medical doctor, continuous control and correction of the photosensitizer supply mode, and in any case manual operation of the laser light irradiation.

It is an object of the present invention to provide a suitable device for PDT of a living organism's tissues by means of activating a flowing suspension of a photosensitizer by light irradiation in every irradiation mode, among others also in pulse mode, during administration of suspension of a photosensitizer to a living organism's tissues.

### SUMMARY OF THE INVENTION

The technical feature of the invention is based on the insight that the device comprises a central control system connected to a suspension supply system and to a control unit of at least one source of light of the optical light exposure system, wherein that central control system enables a simultaneous and separate operation of both systems. The invention is based on the insight that the PDT effect on a living organism's tissues is increased through a general automatic control over both the operation parameters of at least one light source and the parameters of suspension supply. Photosensitizer, which is activated by the light directly during administration, can be brought into natural cavities or into organs/tissues located deeply under skin surface of a living organism, dependent on the purpose of the treatment.

### BRIEF DESCRIPTION OF THE DRAWING

FIG. 1 represents a schematic method of an embodiment of the present device for photo-dynamic therapy of a living organism's tissues.

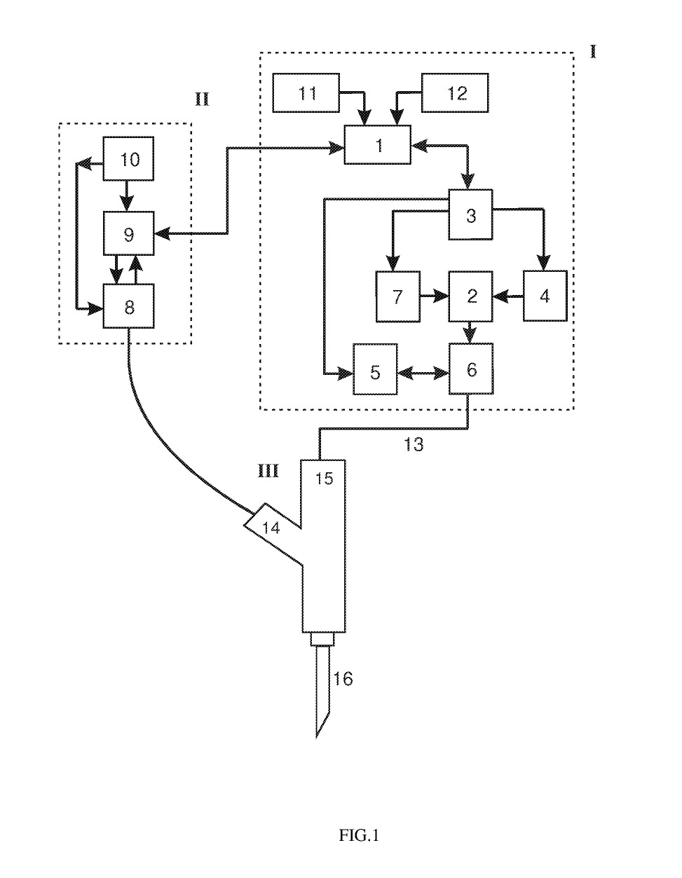

### DETAILED DESCRIPTION OF THE PREFERRED EMBODIMENTS

At least one source of light is used for light irradiation exposure. The at least one source of light is selected from a group including light sources of high power and a large range of wavelength, single LEDs, LEDs in a matrix-arrangement, halogen light sources, laser diodes and laser light sources, and enables irradiating a light having an output power of <20 W and of a wavelength in the range of 380 nm to 1500 nm. Light irradiation frequency can be modified in the range of 0 to 10 Khz, during operation in the impulse mode, the maximum output power amounts to 1 W to 10 kW, frequency modulation in the impulse mode is preferably in the range from 0 to 10 kHz and impulse duration amounts to 100 ns to 10 ms depending on activation needs. The suspension supply system comprises preferably one infusion pump, supplying suspension of photosensitizer at a rate of 100 ml to 2000 ml per hour. Preferably, flow volume and supply rate of suspension of photosensitizer and intensity of the irradiated light are independent of each other. In that way, the efficiency of a photosensitizer's influence on a living organism's tissues is significantly increased.

For injection of suspension of photosensitizer into deeply located tissues, the system for simultaneous delivery and irradiation of suspension of photosensitizer may comprise an injection needle.

A system for simultaneous delivery and irradiation of suspension of photosensitizer may comprise a cannula enabling administration of suspension into natural cavities of a living organism.

Preferably, the suspension of photosensitizer contains nanoparticles of metal oxides and/or silicon oxides of heterocrystal minerals. Active oxygen forms are generated directly at the moment of administration of suspension into a target area of a living organism. Due to precise administration of a photosensitizer and simultaneous activation thereof by light irradiation, healthy tissues are neither damaged nor stressed. The device is used for treatment of ulcers, malignant tumors or may be applied in aesthetic therapy.

A system for simultaneous target supply and irradiation of suspension of photosensitizer contains a Y-shaped body comprising two arms flowing into a main channel, wherein one arm is connected to suspension supply system and the other arm is coupled to an optical light exposure system.

One arm of the Y-shaped body is preferably connected to an infusion pump of the suspension supply system.

The other arm of the Y-shaped body is preferably coupled to the optical light exposure system through a light guide.

The device comprises an optical light exposure system (I), a suspension supply system (II) and a system (III) for simultaneous delivery and irradiation of suspension of photosensitizer.

Optical light exposure system (I) comprises at least one source of light (2), which provides for light irradiation and wavelength. At least one source of light (2) sends light through a suitably selected optical instrument (6) into a light guide (13) to system (III) for simultaneous delivery and irradiation of suspension of photosensitizer. Through optical instrument (6), rays of light from at least one source of light (2) are focused into a bore of light guide (13) of e.g. 600 μm. Power supply system (7) provides electric power to at least one source of light (2) and its control unit (3). On the one side, control unit (3) is connected to central control system (1) of optical light exposure system (I) and obtains also data from power supply system (7) of at least one source of light (2). Control unit (3) of at least one source of light (2) is also connected to a cooling unit (4) for at least one source of light (2). Cooling unit (4) may comprise a Peltier-element and ventilators. Control unit (3) is additionally connected to a control sensor (5) of optical fiber. Control sensor (5) controls and sets up an operation voltage and determinates parameters of light irradiation focused through optical instrument (6).

Central control system (1) of optical light exposure system (I) is further connected to power unit (11) and to data input system (12) for introduction of operation and working parameters. Optical light exposure system (I) is coupled through relative data channels to suspension supply system (II).

Suspension supply system (II) comprises a control unit (9), which is connected to a source of power (10). Control unit (9) is connected to infusion pump (8) in a way that it enables simultaneous or separate supply of a desired volume of suspension of photosensitizer into system (III) for simultaneous delivery and irradiation of the suspension.

System (III) for simultaneous delivery and irradiation of the suspension of photosensitizer consists per se of a Y-shaped body comprising two arms (14, 15) flowing into one main channel. One arm (14) is connected to suspension supply system (II), in a special way coupled to infusion pump (8). The other arm (15) of Y-shaped body is coupled through a light guide (13) to optical light exposure system (I). Main channel of system (III) ends with an injection needle (16) enabling injection of suspension into deeply located tissues or with a cannula enabling delivery of suspension into natural cavities of a living organism, respectively.

The present device for photo-dynamic therapy is operated by central control system (1) through parameters input through a data input system (12), e.g. a touch screen. Central control system (1) is connected to control unit (9) of suspension supply system (II) and to control unit (3) of a source of light (2). The device of the invention enables an optimal treatment of tissues of a living organism during administration of an activated photosensitizer as well as providing for a separate operation of both systems—optical light exposure system (I) and suspension supply system (II).

1. Device for photo-dynamic therapy of a living organism's tissues comprising:
an optical light exposure system including at least one source of light selected from a group including light sources of high power and large range of wavelength, single LEDs, LEDs in a matrix-arrangement, halogen light sources, laser diodes and laser light sources,
a suspension supply system, and
a unit for simultaneous delivery and irradiation of a suspension of a photosensitizer, wherein a central control system is connected to the suspension supply system and to a control unit of said at least one source of light of the optical light exposure system.

2. The device according to claim 1, wherein said at least one source of light enables irradiation of light of up to 20 W power and of a wavelength in a range between 380 nm and 1500 nm.

3. The device according to claim 1, wherein the suspension supply system comprises an infusion pump, enabling delivery of suspension at a rate of 100 ml to 2000 ml per hour.

4. The device according to claim 2, wherein the suspension supply system comprises an infusion pump, enabling delivery of suspension at a rate of 100 ml to 2000 ml per hour.

5. The device according to claim 1, wherein the unit for simultaneous delivery and irradiation of suspension of photosensitizer comprises an injection needle enabling a targeted delivery of suspension of photosensitizer into deeply situated tissues of a living organism.

6. The device according to claim 2, wherein the unit for simultaneous delivery and irradiation of suspension of photosensitizer comprises an injection needle enabling a targeted delivery of suspension of photosensitizer into deeply situated tissues of a living organism.

7. The device according to claim 3, wherein the unit for simultaneous delivery and irradiation of suspension of photosensitizer comprises an injection needle enabling a targeted delivery of suspension of photosensitizer into deeply situated tissues of a living organism.

8. The device according to claim 1, wherein the system for simultaneous delivery and irradiation of suspension of photosensitizer comprises a cannula enabling a targeted delivery of suspension of photosensitizer into natural cavities of a living organism.

9. The device according to claim 2, wherein the system for simultaneous delivery and irradiation of suspension of photosensitizer comprises a cannula enabling a targeted delivery of suspension of photosensitizer into natural cavities of a living organism.

10. The device according to claim 3, wherein the system for simultaneous delivery and irradiation of suspension of photosensitizer comprises a cannula enabling a targeted delivery of suspension of photosensitizer into natural cavities of a living organism.

11. The device according to claim 1, wherein the suspension of photosensitizer contains nanoparticles of metal oxides and/or silicon oxides of heterocrystal minerals.

12. The device according to claim 2, wherein the suspension of photosensitizer contains nanoparticles of metal oxides and/or silicon oxides of heterocrystal minerals.

13. The device according to claim 3, wherein the suspension of photosensitizer contains nanoparticles of metal oxides and/or silicon oxides of heterocrystal minerals.

14. The device according to claim 1, wherein the system for simultaneous delivery and irradiation of suspension of photosensitizer comprises a Y-shaped body including first and second arms flowing into one main channel, wherein the first arm is connected to suspension supply system and the second arm is coupled to optical light exposure system.

15. The device according to claim 2, wherein the system for simultaneous delivery and irradiation of suspension of photosensitizer comprises a Y-shaped body including first and second arms flowing into one main channel, wherein the first arm is connected to suspension supply system and the second arm is coupled to optical light exposure system.

16. The device according to claim 3, wherein the system for simultaneous delivery and irradiation of suspension of photosensitizer comprises a Y-shaped body including first and second arms flowing into one main channel, wherein the first arm is connected to suspension supply system and the second arm is coupled to optical light exposure system.

17. The device according to claim 5, wherein the system for simultaneous delivery and irradiation of suspension of photosensitizer comprises a Y-shaped body including first and second arms flowing into one main channel, wherein the first arm is connected to suspension supply system and the second arm is coupled to optical light exposure system.

18. The device according to claim 8, wherein the system for simultaneous delivery and irradiation of suspension of photosensitizer comprises a Y-shaped body including first and second arms flowing into one main channel, wherein the first arm is connected to suspension supply system and the second arm is coupled to optical light exposure system.

19. The device according to claim 16, wherein the first arm of Y-shaped body is connected to infusion pump of suspension supply system.

20. The device according to claim 16, wherein the second arm of Y-shaped body is coupled to optical light exposure system through a light guide.
